(19) United States
(12) Patent Application Publication
(10) Pub. No.: US 2002/0188022 A1
(43) Pub. Date: Dec. 12, 2002

(54) Methods for modulation, stimulation, and inhibition of glutamate reuptake

(75) Inventors: Maria-Luisa Maccecchini, West Chester, PA (US); Xue-Feng Pei, Lansdale, PA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Annovis. Inc.

(21) Appl. No.: 10/021,177

(22) Filed: Oct. 30, 2001

Related U.S. Application Data
(60) Provisional application No. 60/244,252, filed on Oct. 30, 2000.

(51) Int. Cl. A61K 31/198; A61K 31/22

(52) U.S. Cl. 514/551; 514/561

(57) ABSTRACT

Disclosed is a method for inhibiting, stimulating, modulating, or regulating glutamate reuptake. The method makes use of compounds that are ligands of glutamate receptors, including many agonists, or antagonists of glutamate receptors. It has been discovered that such compounds can bind to or modulate glutamate transporters and affect extracellular glutamate levels by affecting transporter activity. The disclosed compounds can have a variety of effects on glutamate transporter activity including activation or inhibition. Such compounds are useful to treat various neurological diseases and conditions involving glutamate transporter and glutamate receptor activation. For example, excess extracellular glutamate is a cause of excessive activation of glutamate receptors. Stimulating glutamate reuptake by glutamate transporters can ameliorate excessive activation of glutamate receptors by reducing the extracellular glutamate concentration. Prodrug forms of transporter compounds can be used as drugs.

### CROSS REFERENCE TO RELATED APPLICATIONS

Priority is claimed to U.S. Provisional application Serial No. 60/244,252, filed Oct. 30, 2000, the teachings of which are incorporated herein.

### BACKGROUND OF THE INVENTION

This invention generally relates to glutamate transporters and more specifically to methods for inhibiting, stimulating, and modulating glutamate reuptake.

During the past twenty-five years, a revolution in understanding the basic structure and chemistry of the synaptic interconnections of neural tissues has taken place, which has yielded knowledge relevant to the treatment of neural tissue damage and disorders. The studies have centered around an understanding of the properties of the neurochemical transmitters released from presynaptic membranes and, most importantly, the postsynaptic receptors for these transmitters. During the past ten years, a great deal of attention has been directed to the excitatory amino acids (EAAs), principally glutamic acid (the primary excitatory neurotransmitter) and aspartic acid, and their receptors since these amino acids mediate the fast excitatory transmission in the mammalian central nervous system. Thus, glutamic acid can bring about changes in the postsynaptic neuron that reflect the strength of the incoming neural signals.

Two major classes of EAA receptors are distinguished: ionotropic and metabotropic. The ionotropic receptors contain ligand-gated ion channels and mediate ion fluxes for signaling, while the metabotropic receptors use G-proteins for signaling. Further sub-classification of the ionotropic EAA glutamate receptors is based upon the agonists (stimulating agents) other than glutamic and aspartic acid that selectively activate the receptors. Presently, it is believed that there are three major subtypes of ionotropic glutamate receptors based on binding at defined concentrations: 1) a receptor responsive to N-methyl-D-aspartate (NMDA); 2) a receptor responsive to α-amino-3-hydroxy-5-methyl-4-isoxazolepropionic acid (AMPA); and 3) a receptor responsive to KA. The NMDA receptor controls the flow of both divalent (Ca⁺⁺) and monovalent (Na⁺, K⁺) ions into the postsynaptic neural cell although it is the Ca⁺⁺ flux which is of the greatest interest. The AMPA and KA receptors also regulate the flow into postsynaptic cells of monovalent K⁺ and Na⁺ and occasionally divalent Ca⁺⁺. The metabotropic EAA receptors are divided into three sub-groups that are unrelated to ionotropic receptors, and are coupled via G-proteins to intracellular second messengers. These metabotropic EAA receptors are classified based on receptor homology and second messenger linkages, and there continue to be reports of novel metabotropic EAA receptors.

EAA receptors have been implicated during development in specifying neuronal architecture and synaptic connectivity and may be involved in experience-dependent synaptic modifications. These receptors have also drawn interest since they appear to be involved in a broad spectrum of CNS disorders. For example, during brain ischemia caused by stroke or traumatic injury, excessive amounts of the EAA glutamic acid are released from damaged or oxygen deprived neurons. Binding of this excess glutamic acid to the postsynaptic glutamate receptors opens their ligand-gated ion channels, thereby allowing an ion influx which in turn activates a biochemical cascade resulting in protein, nucleic acid and lipid degradation and cell death. This phenomenon, known as excitotoxicity, is also thought to be responsible for the neurological damage associated with other disorders ranging from hypoglycemia, ischemia, and epilepsy to chronic neurodegeneration in Huntington's, Parkinson's, and Alzheimer's diseases.

L-Glutamate (Glu) is the major excitatory neurotransmitter in the central nervous system (CNS) and acts at distinct subtypes of ionotropic (iGluRs) and metabotropic (mGluRs) receptors. Excessive activation of iGluRs and group I mGluRs may result in neuronal death. Many neurodegenerative conditions, including Parkinson's disease, Alzheimer's disease, cerebral ischaemia, epilepsy, Huntington's chorea and amyotrophic lateral sclerosis (ALS), have been linked to disturbed Glu homeostasis (Lipton and Rosenberg, New Eng. J. Med., 330: 613-622 (1994); Gegelashvili and Schousboe, Mol. Pharmacol., 52: 6-15 (1997); Robinson and Dowd, Adv. Pharmacol,., 37: 69-115 (1997)). Extracellular Glu concentrations are maintained within physiological levels exclusively by Glu transporters (GluTs; also known as amino acid transporters), since no extracellular enzymes exist for the breakdown of Glu (Robinson and Dowd, 1997). Thus, GluTs are responsible for the high-affinity uptake of extracellular Glu. They permit normal excitatory transmission as well as protection against excitotoxicity (Robinson and Dowd, 1997). The abundance and function of GluTs is therefore clearly relevant to neurodegenerative diseases. Overall, Glu interacts with various proteomic binding sites, be they receptors or membrane transporters. On the basis of pharmacological studies using conformationally restricted molecules it is clear that these interactions involve multiple conformations of Glu. A number of compounds acting on the ionotropic EAA receptors have been described.

Since 1992, molecular cloning and molecular biological studies have identified five subtypes of GluT (nomenclature in human EAAT1-5) demonstrating discrete cellular and regional localizations as well as distinct molecular and pharmacological characteristics. GLAST (the rodent homologue of the human EAAT1) is predominantly located in cerebellar Bergmann glia, but is also present in glial cells throughout the CNS and in a small number of neurones under certain conditions. GLT1 (EAAT2) is almost exclusively glial and is widespread and abundant throughout the forebrain, cerebellum and spinal cord. The transporters EAAC1 (EAAT3) and EAAT4 are found predominantly in neurones, with EAAC1 being abundantly expressed throughout the brain and in spinal cord, and EAAT4 unique to Purkinje cells of the cerebellum. EAAT5 is expressed predominantly in the retina, but its cellular specificity is yet to be determined. The majority of synapses in the CNS are in close apposition with glial cells, and glial GluTs appear to be responsible for most Glu transport in the CNS.

Comparatively little is known about the various glutamate transporters. A principal reason for this lack of knowledge is that few compounds are known which selectively bind to individual subtypes of glutamate transporters, and also because the interpretation of the pharmacology of these compounds is generally complicated by concurrent direct actions at EAA receptors.

Many compounds are known to bind to EAATs and inhibit transporter function. Inhibitors of EAATs fall into two major classes that differ in their mode of action: non-transportable blockers and competitive substrates. Most available inhibitors are competitive substrates, which are generally transported at a slower rate than Glu and may displace cytoplasmic Glu through heteroexchange, leading to further extracellular accumulation of Glu.

In addition to direct actions on transport, EAATs are subject to complex modulation by a variety of compounds, including glutamate receptor agonists, zinc, arachidonic acid, cyclic AMP and nitric oxide, which appear to effect the expression, mobilization and function of EAATs.

Chemists and pharmacologists have attempted to understand the critical aspects of shape, pharmacophore position and pharmacophore type that are important for positive or negative modulation of the transporters. Generally random screening and hit or miss synthesis and testing were used to find new modulators for the transporters. It is likely that each glutamic acid transporter subtype, such as GLAST and GLT1, will have its own requirements and pharmacophore positions. The optimal way to design new non-transportable blockers and competitive substrates is to have a non-transportable blocker and/or competitive substrate model for each transporter subtype that contains the specific shape and pharmacophore positions and then to use this model to link the pharmacophores into novel molecules.

Much of what has been learned about glutamate receptors has been made possible by the discovery of compounds which block one or another action of the various modulatory agents. The approach of using blocking agents to map pathways has a long history in biochemical and biophysical research and very often these blocking agents have been discovered to be useful therapeutic agents. A similar approach, although applicable to glutamate transporters, cannot be used in the absence of compounds which selectively bind to a particular glutamate transporter subtype. It would be useful to have compounds that specifically stimulate, inhibit, or modulate particular subtypes of glutamate transporters.

It is therefore an object of the present invention to provide a method of selectively inhibiting, stimulating, modulating, or regulating glutamate reuptake.

It is a further object of the present invention to provide compounds which selectively inhibit, stimulate, modulate, or regulate glutamate reuptake.

### BRIEF SUMMARY OF THE INVENTION

Disclosed is a method for inhibiting, stimulating, modulating, or regulating glutamate reuptake. The method makes use of compounds that are ligands, agonists, or antagonists of glutamate receptors. It has been discovered that many such compounds can bind to glutamate transporters and affect extracellular glutamate levels by affecting transporter activity. The disclosed compounds can have a variety of effects on glutamate transporter activity including activation or inhibition. Such compounds are useful to treat various neurological diseases and conditions involving glutamate transporter and glutamate receptor activation. For example, excess extracellular glutamate is a cause of excessive activation of glutamate receptors. Stimulating glutamate reuptake by glutamate transporters can ameliorate excessive activation of glutamate receptors by reducing the extracellular glutamate concentration. Inhibiting glutamate reuptake by glutamate transporters can ameliorate insufficient activation of glutamate receptors by increasing the extracellular glutamate concentration. This could be useful for enhancing learning and memory in, for example, neurodegenerative disorders such as Alzheimer's disease. Prodrug forms of transporter compounds can be used as drugs.

### BRIEF DESCRIPTION OF THE DRAWINGS

FIG. 1 is a graph of association of [³H]-4MG to homogenized brain tissue (in percent specific binding) versus time (in minutes).

FIG. 2 is a graph of dissociation of [³H]-4MG to homogenized brain tissue (in percent specific binding) versus time (in minutes).

FIG. 3 is a graph of inhibition of binding of [³H]-4MG to homogenized brain tissue (in percent specific binding) versus concentration of various transporter compounds in molar units.

FIG. 4 is a graph of dissociation of [³H]-4MG to homogenized brain tissue (in percent specific binding) versus concentration of various transporter compounds in molar units.

FIG. 5 is a graph of specific binding (in percent control) of [³H]-D-aspartate and [³H]-4MG in the presence of various transporter and receptor compounds.

FIGS. 6A-6I are diagrams of the structures of examples of transporter compounds.

FIG. 7 shows the structures of compounds in Table 2 with high affinity for the glial glutamate transporter.

### DETAILED DESCRIPTION OF THE INVENTION

It has been discovered that ligands, agonists, and antagonists of glutamate receptors can bind to, and affect the function of, glutamate transporters. Such compounds, referred to herein as transporter compounds, can bind to glutamate transporters and affect extracellular glutamate levels by affecting transporter activity. The disclosed compounds can have a variety of effects on glutamate transporter activity including activation or inhibition.

The prototype compound, (2S,4R)-4-methylglutamate (4MG), is known to bind low affinity kainate receptors. It was discovered that, in the presence of near-physiological concentrations of the sodium ion, 4MG can bind selectively to glutamate transporters having characteristics of the glial glutamate transporters GLAST and GLT1. It was realized that many of the members of the class of compounds that are ligands of glutamate receptors, including many agonists, or antagonists of glutamate receptors, will have similar properties. It was also realized that such compounds can be used to alter the function of glutamate transporters. Corresponding effects on extracellular glutamate levels and glutamate receptor activation will ameliorate the effects of neurological diseases and conditions involving glutamate transporter and glutamate receptor activation. Thus, the disclosed method makes use of compounds that are ligands, agonists, or antagonists of glutamate receptors to affect glutamate transporter functions.

Glossary of Terms.

The term “activity” as used herein in reference to a glutamate receptor refers to the flow of cations through the receptor. Increased activity means increased flow. Decreased activity means reduced flow. The term “activity” as used herein in reference to a glutamate transporter refers to the transport of excitatory amino acids by the transporter. Increased activity means increased transport. Decreased activity means decreased transport.

The term “activation” as used herein in reference to a glutamate receptor refers to an increase in the flow of cations through the receptor. The term “activation” as used herein in reference to a glutamate transporter refers to increased transport of excitatory amino acids by the transporter. The term “excessive activation” used in reference to a glutamate receptor refers to an activation resulting in the opening of an ion channel for a prolonged period of time so that there is an excessive ion flux through the channel, which results in substantial damages to the cell including cell death.

The term “ligand” as used herein means any compound which binds to a glutamate receptor, and includes but is not limited to agonists, antagonists and partial agonists.

The term “agonist” as used herein means any compound which increases the activity of a glutamate receptor, and which has not been observed to decrease the activity of the same receptor.

The term “antagonist” as used herein means any compound which decreases the activity of a glutamate receptor, and which has not been observed to increase the activity of the same receptor.

The term “partial agonist” as used herein means a compound which modulates the activity of a glutamate receptor or transporter depending on the presence or absence of the principal site modulator(s). In the absence of the principal site modulator(s), a partial agonist increases the activity of the glutamate receptor or transporter but at a lower level than achieved by the principal site modulator(s). A partial agonist partially activates the receptor or transporter. In the presence of the principal site modulator(s), a partial agonist decreases the activity of a glutamate receptor or transporter below the activity normally achieved by the principal site modulator(s).

The term “principal site ligand” as used herein refers to known endogenous ligands binding to a site.

The term “glutamic acid” as used herein means the amino acid L-glutamic acid (“Glu”).

The term “neuropsychopharmacological disorder” as used herein means a disorder resulting from, or associated with, a reduced or excessive flux of ions through a glutamate receptor ligand-gated cation channel or other effects of EAAT inhibition including impairment of intermediary metabolism involving neurons and glia, and includes cognitive, learning, and memory deficits, chemical toxicity (including substance tolerance and addiction), excitotoxicity, neurodegenerative disorder (such as Huntington's disease, Parkinson's disease, amyotrophic lateral sclerosis and Alzheimer's disease), post-stroke sequelae, epilepsy, seizures, mood disorders (such as bipolar disorder, dysthymia, anxiety, and seasonal effective disorder), and depression. Neurodegenerative disorders can result from dysfunction or malfunction of the receptor and/or transporter. As used herein, this term includes pain.

The term “potency” as used herein refers to the molar concentration at which a specified effect on a receptor channel or transporter activity is observed. Specifically, potency for a compound exhibiting antagonistic effect is presented as the IC₅₀ value, which is the concentration at which inhibition of activity is 50% of the maximum inhibition achievable. Lower values indicate higher potency. Potency for a compound exhibiting agonistic effect is presented as the EC₅₀ value, which is the concentration at which enhancement of activity is 50% that of the maximum enhancement achievable. Lower values indicate higher potency.

The term “efficacious” as used herein refers to a comparison of the maximum increase or decrease in activity achieved by a particular compound with maximum increase or decrease in activity achieved by a principal site ligand. Efficacy refers to magnitude of a specified effect.

The term “pharmacophore” as used herein means an atom or group of atoms that electrostatically or through hydrogen bonds interacts directly with the receptor or transporter protein.

The term “specifically binds” as used herein means a compound binding to a receptor or transporter with an affinity at least three times as great as a compound which binds to multiple sites, receptors, or transporters.

The term “prodrug” as used herein means a compound that is converted into a bioactive form in an animal body. The prodrug itself may or may not have a bioactivity.

### Materials

The disclosed method can be carried out, and the identified compounds can be used, by employing the following materials as described below and elsewhere herein.

A. Transporter Compounds

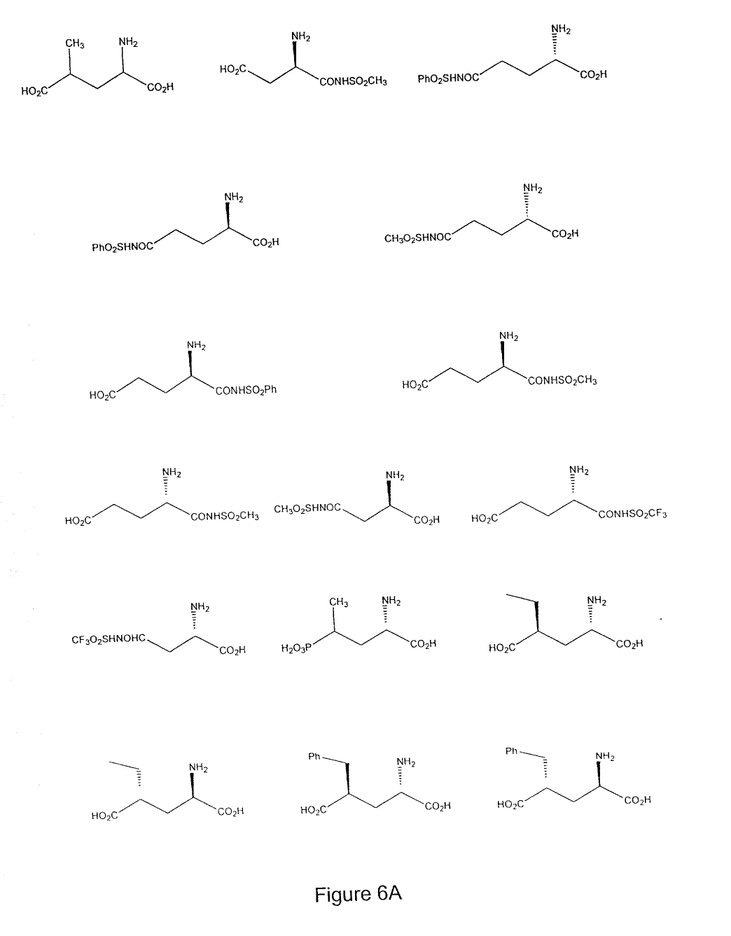

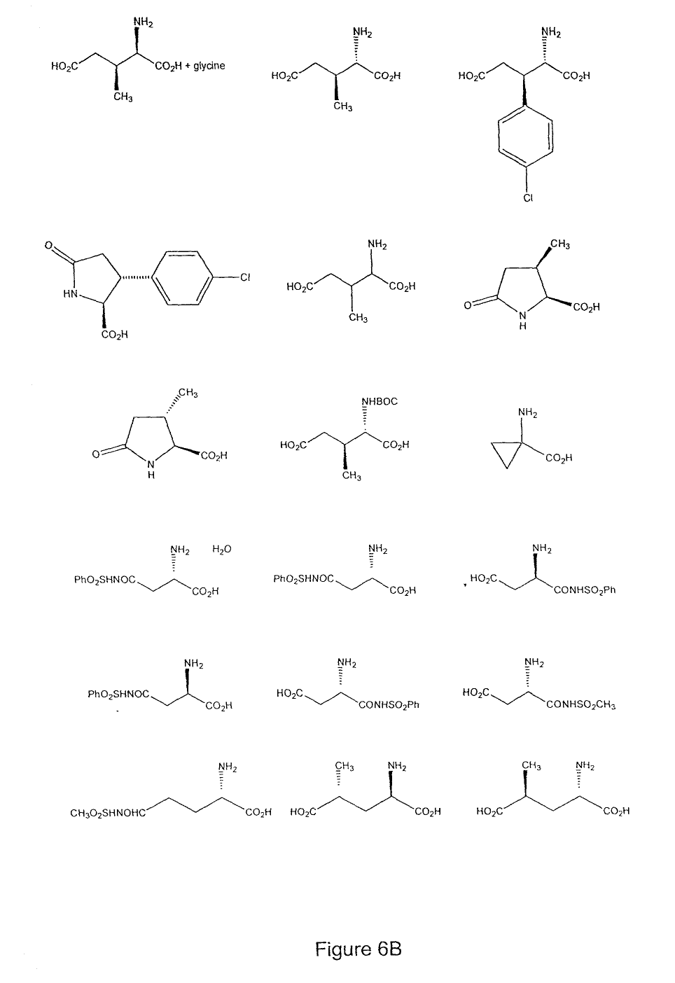

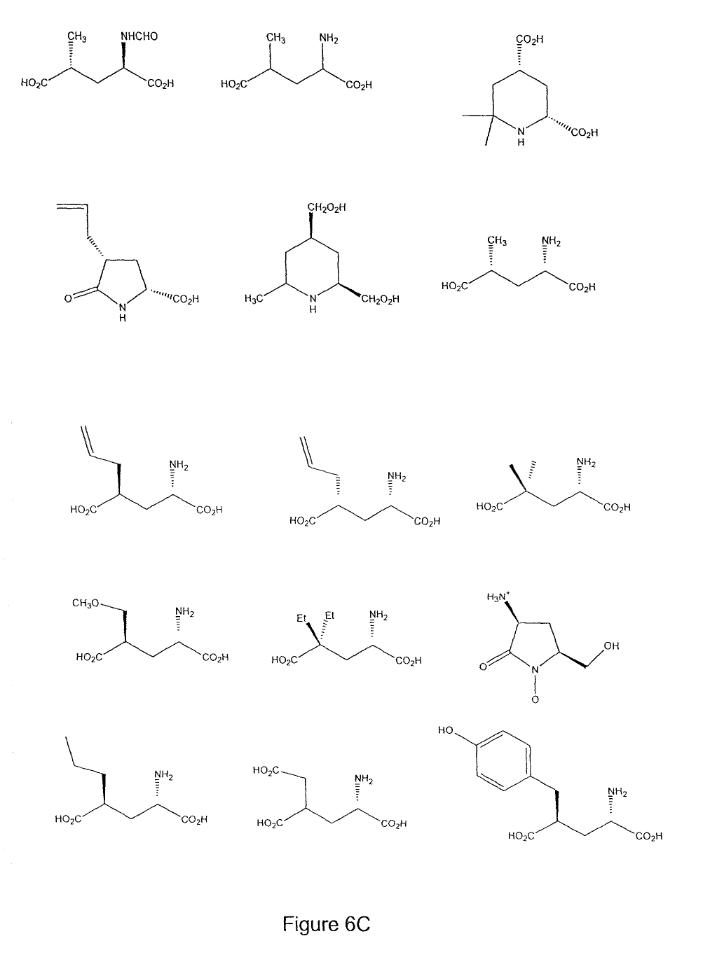

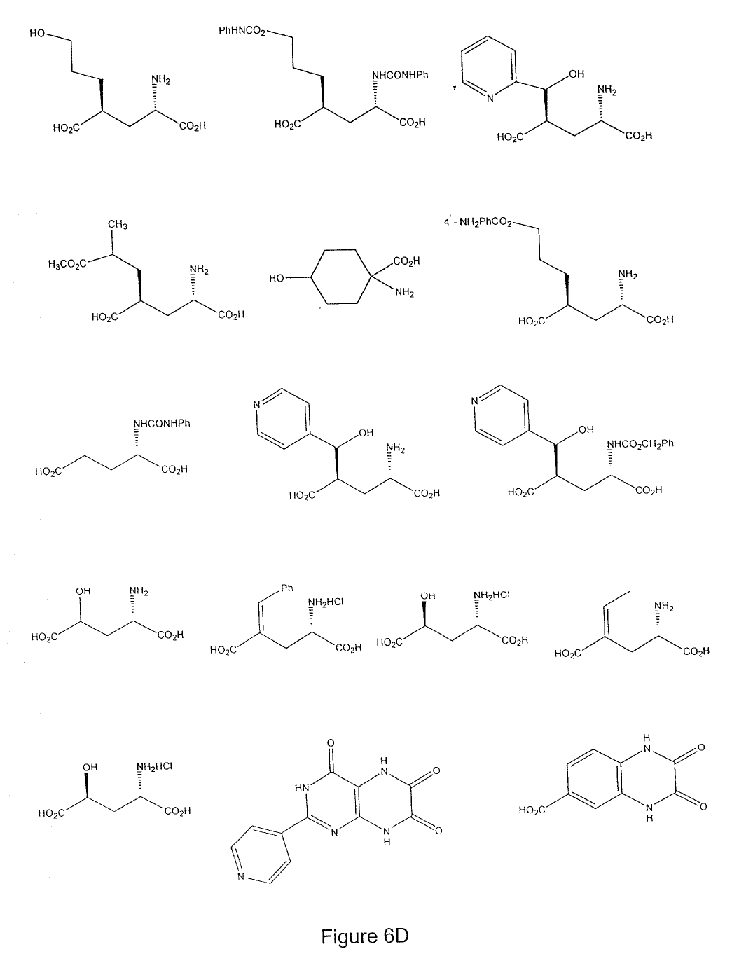

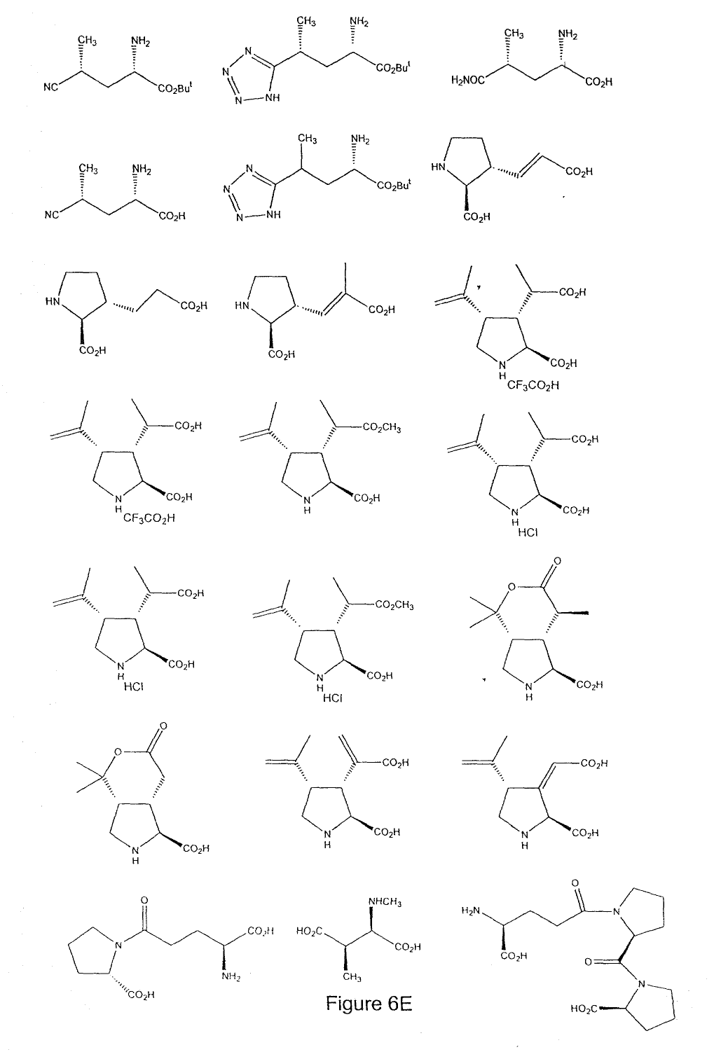

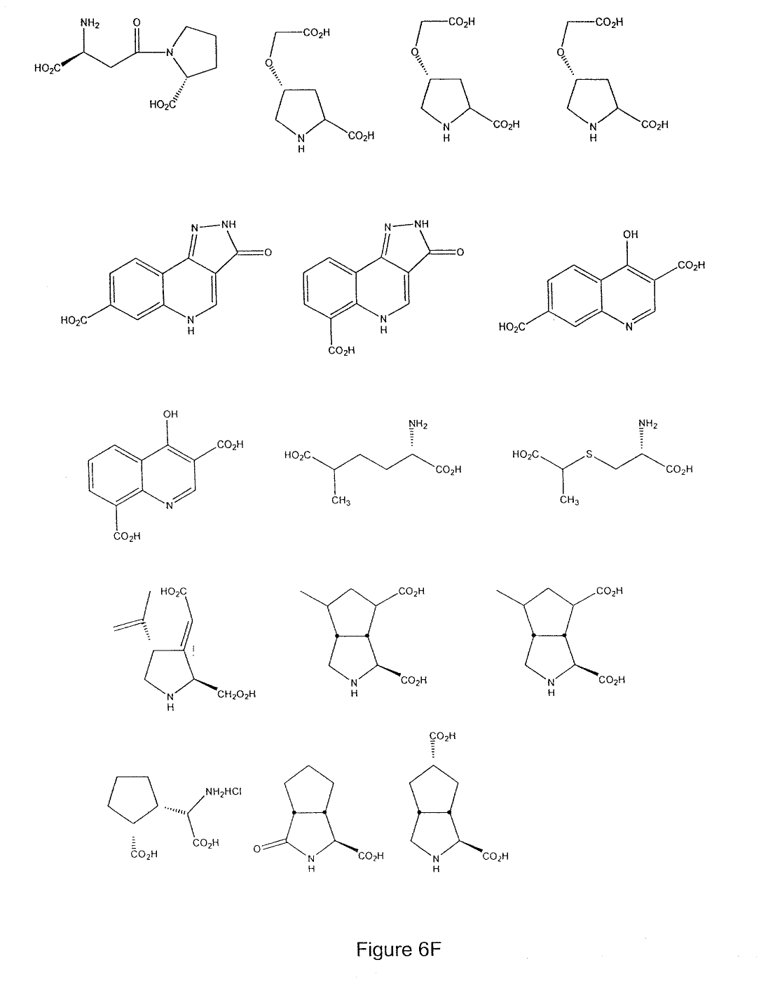

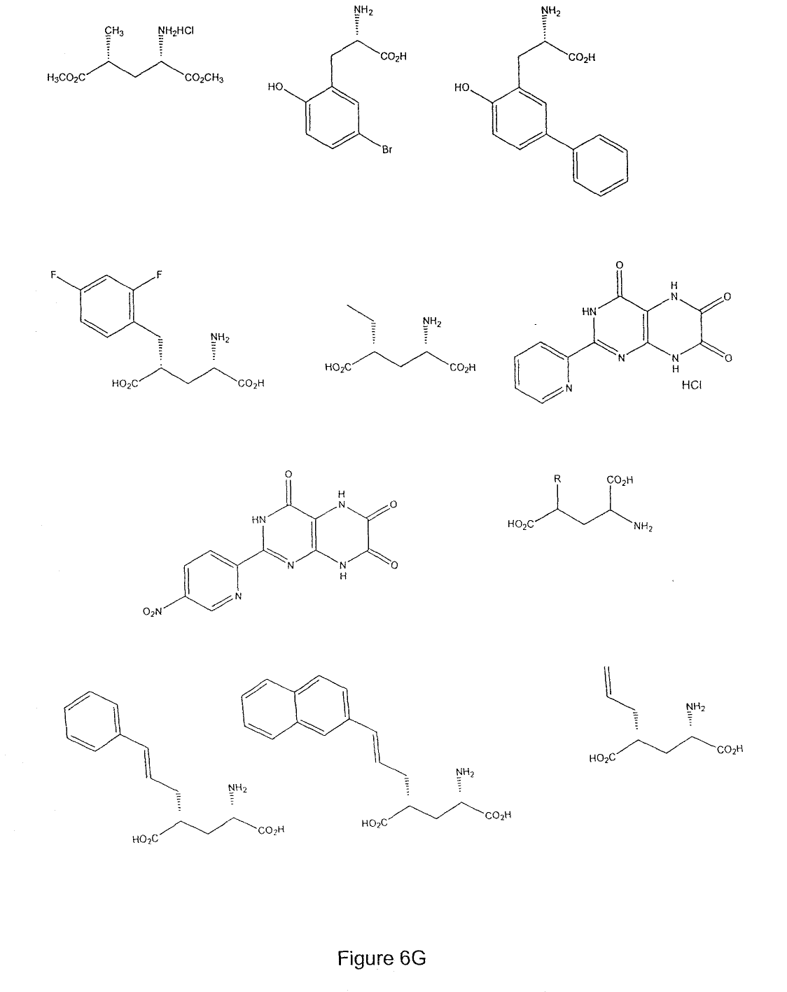

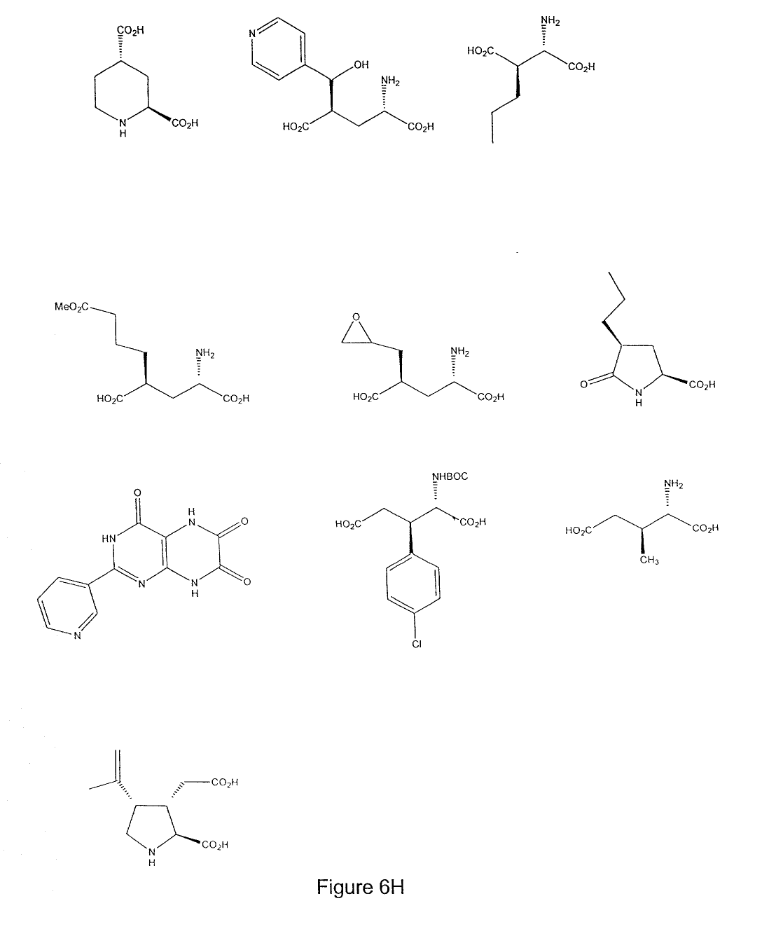

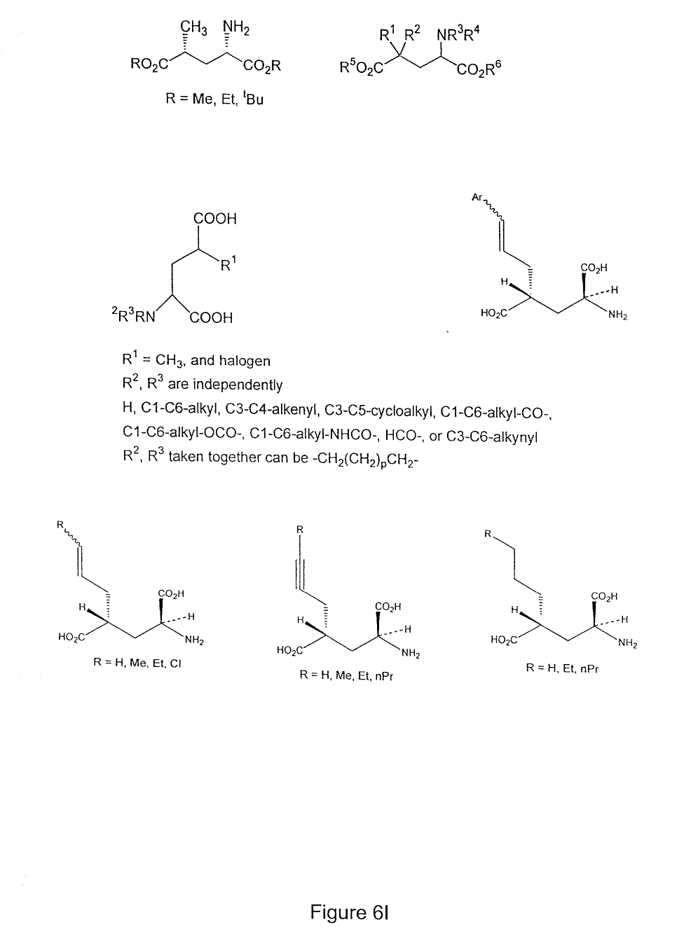

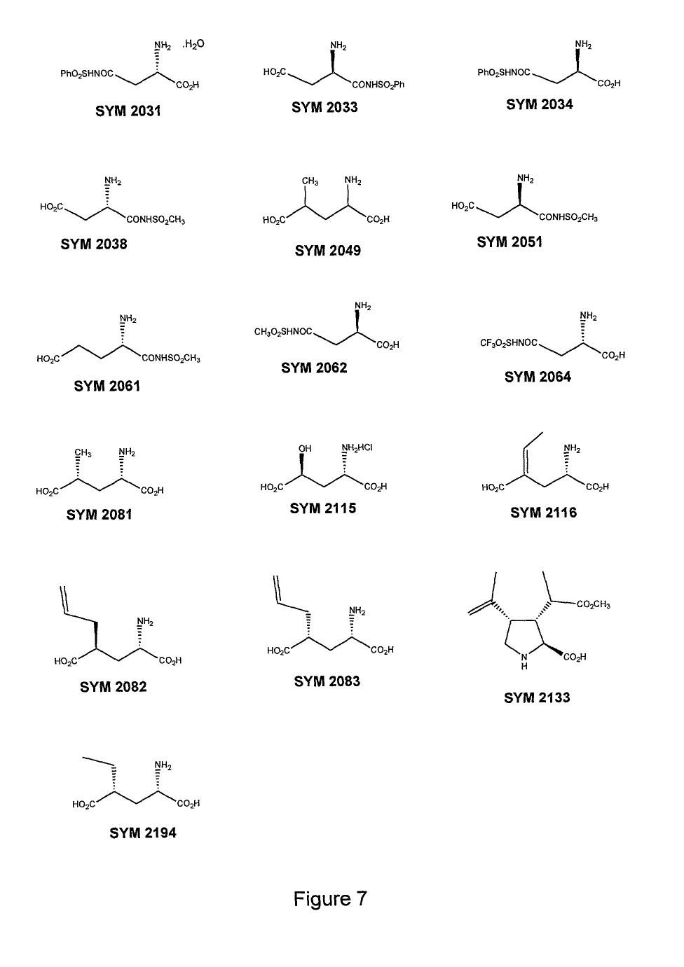

A transporter compound is a compound that binds to one or more types of glutamate transporters. Preferred transporter compounds are compounds that are agonists or antagonists of glutamate receptors. Many such compounds are known. Examples of transporter compounds are shown in FIGS. 6, 7 and Table 2. The transporter compounds are used in the disclosed method to inhibit, stimulate, modulate, or regulate glutamate transporter activity. Transporter compounds can have a variety of effects on glutamate transporter activity including activation or inhibition. These compounds are expected to affect or interfere with glutamate reuptake by the glutamate transporter and thus can be used to modulate, stimulate, or inhibit glutamate reuptake. Such compounds are useful to treat various neurological diseases and conditions involving glutamate transporter and glutamate receptor activation. For example, excess extracellular glutamate is a cause of excessive activation of glutamate receptors. Stimulating glutamate reuptake by glutamate transporters can ameliorate excessive activation of glutamate receptors by reducing the extracellular glutamate concentration.

In one preferred embodiment, the transporter compound has the structure

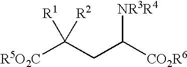

wherein

R¹, R², R⁵ and R⁶ are independently

1) C1-C6-alkyl,

2) C3-C4-alkenyl,

3) C3-C5-cycloalkyl,

4) H, or

5) halogen;

R³ and R⁴ are independently

1) H,

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) C1-C6-alkyl-SO₂—

9) CF₃SO₂—

10) PhSO₂—

11) HCO—, or

12) C3-C6-alkynyl; and

R³ and R⁴ taken together can be—CH₂(CH₂)ₙCH₂—wherein n is 0, 1, 2, or 3.

In another preferred embodiment, the transporter compound has the structure

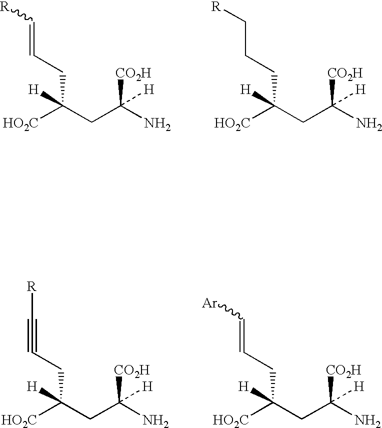

wherein R═H, C1-C6-alkyl, C3-C4-alkenyl, C3-C5-cycloalkyl, C1-C6-alkyl-CO—, C1-C6-alkyl-OCO—, C1-C6-alkyl-NHCO—, HCO—, or C3-C6-alkynyl.

In a further preferred embodiment, the transporter compound has the structure

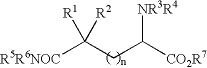

wherein

n is an integer selected from the group consisting of 0, 1, 2, and 3;

R¹, R², R⁵ and R⁷ are independently

1) C1-C6-alkyl,

2) C3-C4-alkenyl,

3) C3-C5-cycloalkyl,

4) H, or

5) halogen;

R³ and R⁴ are independently

1) H,

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) C1-C6-alkyl-SO₂—

9) CF₃SO₂—

10) PhSO₂—

11) HCO—, or

12) C3-C6-alkynyl;

R³ and R⁴ taken together can be—CH₂(CH₂)ₘCH₂—wherein m is 0, 1, 2, or 3;

R⁶ is independently

1) H,

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) C1-C6-alkyl-SO₂—

9) CF₃SO₂—

10) PhSO₂—

11) HCO—, or

12) C3-C6-alkynyl; and

R⁵ and R⁶ taken together can be—CH₂(CH₂)ₖCH₂—wherein k is 0, 1, 2, or 3.

In one most preferred embodiment, the transporter compound has the structure

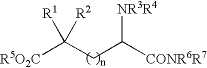

wherein

n is an integer selected from the group consisting of 0, 1, 2, and 3;

R¹, R², R⁵ and R⁷ are independently

1) C1-C6-alkyl,

2) C3-C4-alkenyl,

3) C3-C5-cycloalkyl,

4) H, or

5) halogen;

R³ and R⁴ are independently

1) H,

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) C1-C6-alkyl-SO₂—

9) CF₃SO₂—

10) PhSO₂—

11) HCO—, or

12) C3-C6-alkynyl;

R³ and R⁴ taken together can be—CH₂(CH₂)ₘCH₂—wherein m is 0, 1, 2, or 3.

R⁶ is independently

1) H,

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) C1-C6-alkyl-SO₂—

9) CF₃SO₂—

10) PhSO₂—

11) HCO—, or

12) C3-C6-alkynyl; and

R⁶ and R⁷ taken together can be—CH₂(CH₂)ₖCH₂—wherein k is 0, 1, 2, or 3.

Any of the transporter compounds can be made in prodrug form. In general, an additional moiety is added to the compound via an ester, carbonate or amine bond. Such bonds can be broken in vivo resulting in generation of the active form of the compound.

C. Synthesis

The compounds to be used in the disclosed method may be prepared using synthetic reactions and techniques available in the art, as described, for example in March, “Advanced Organic Chemistry,” 4^{th} Edition, 1992, Wiley-Interscience Publication, New York. The reactions are performed in solvents suitable to the reagents and materials employed and suitable for the transformation being effected. Depending upon the synthetic route selected, and the functionality of the starting material or intermediates, the appropriate protection groups and deprotection conditions available in the art of organic synthesis may be utilized in the synthesis of the compound. It is understood by those skilled in the art of organic synthesis that the functionality present on the molecule must be consistent with the chemical transformations proposed. This will frequently necessitate judgment as to the order of synthetic steps, protecting groups required, deprotection conditions and generation of enolate to enable attachment of appropriate groups on the molecule.

D. Pharmaceutical Compositions

A. Effective Dosage Ranges

Transporter compounds can be administered parenterally, either subcutaneously, intramuscularly, or intravenously, or alternatively, administered orally in a dose range of between approximately 0.5 mg/kg body weight and 150 mg/kg body weight.

B. Carriers and Additives

Transporter compounds can be administered parenterally, in sterile liquid dosage forms. In general, water, a suitable oil, saline, aqueous dextrose, and related sugar solutions and glycols such as propylene glycol or polyethylene glycols are suitable carriers for parenteral solutions. Solutions for parenteral administration preferably contain a water soluble form of the active ingredient, suitable stabilizing agents, and, if necessary, buffer substances.

Antioxidizing agents such as sodium bisulfite, sodium sulfite, or ascorbic acid, either alone or in combination, can be used as suitable stabilizing agents. Also used are citric acid and its salts and sodium EDTA. In addition, parenteral solutions can contain preservatives, such as benzalkonium chloride, methyl- or propylparaben, and chlorobutanol.

Transporter compounds can be administered orally in solid dosage forms, such as capsules, tablets and powders, or in liquid dosage forms, such as elixirs, syrups, and suspensions. Gelatin capsules contain the active ingredient and powdered carriers, such as lactose, starch, cellulose derivatives, magnesium stearate, or stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere.

Other agents that can be used for delivery include liposomes, microparticles (including microspheres and microcapsules), and other release devices and forms that provide controlled, prolonged or pulsed, delivery or which enhance passage through the blood brain barrier, for example.

Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6,:275-283 (1987); and Mathiowitz, et al., J. Appl. Polymer Sci. 35:755-774 (1988). The selection of the method depends on the polymer selection, the size, external morphology, and crystallinity that is desired, as described, for example, by Mathiowitz, et al., Scanning Microscopy 4:329-340 (1990); Mathiowitz, et al., J. Appl. Polymer Sci. 45:125-134 (1992); and Benita, et al., J. Pharm. Sci. 73:1721-1724 (1984). Methods routinely used by those skilled in the art include solvent evaporation, hot melt encapsulation, solvent removal, spray drying, phase separation and ionic crosslinking of gel-type polymers such as alginate or polyphosphazines or other dicarboxylic polymers to form hydrogels.

Other delivery systems including films, coatings, pellets, slabs, and devices can be fabricated using solvent or melt casting, and extrusion, as well as standard methods for making composites.

The microparticles can be suspended in any appropriate pharmaceutical carrier, such as saline, for administration to a patient. In the most preferred embodiment, the microparticles will be stored in dry or lyophilized form until immediately before administration. They will then be suspended in sufficient solution for administration. The polymeric microparticles can be administered by injection, infusion, implantation, orally, or administration to a mucosal surface, for example, the nasal-pharyngeal region and/or lungs using an aerosol, or in a cream, ointment, spray, or other topical carrier, for example, to rectal or vaginal areas. The other devices are preferably administered by implantation in the area where release is desired. The materials can also be incorporated into an appropriate vehicle for transdermal delivery as well as stents. Appropriate vehicles include ointments, lotions, patches, and other standard delivery means.

When an alkyl substituent is identified herein, the normal alkyl structure is intended (for example, butyl is n-butyl) unless otherwise specified. However, when radicals are identified (for example, R¹), both branched and straight chains are included in the definition of alkyl, alkenyl, and alkynyl.

Pharmaceutically acceptable salts include both the metallic (inorganic) salts and organic salts; a list of which is given in Remington's Pharmaceutical Sciences 17th Edition, p. 1418 (1985). It is well known to one skilled in the art that an appropriate salt form is chosen based on physical and chemical stability, flowability, hydroscopicity and solubility.

Depending on the required activity, the disclosed compounds may be used as pharmaceutical neuroprotectants to treat acute cases of CNS injury and trauma as well as to treat convulsions, mood disorders, alleviation of pain, and other neuropsychiatric and neurodegenerative diseases due, in part, to chronic disturbances in the ion flux through glutamate receptors or in glutamate transport (such as disturbances in glutamate reuptake). Modulation of glutamate transporter activity can be used to treat both conditions resulting from a dysfunction in glutamate transport and conditions resulting from dysfunction of glutamate receptors. In the later category, for example, are conditions resulting from excessive activation of a glutamate receptor even in the presence of a physiological level of extracellular glutamate. Lowering the level of extracellular glutamate (by, for example, increasing glutamate reuptake) can reduce activation of the receptor.

Transporter compounds can be selected for the required activity to treat the relevant disorder. As used herein, the common definitions of neuropsychiatric and neurodegenerative disorders are intended, where diagnosis is based on the alleviation of abnormal behavior, rather than histopathology.

The ester, carbonate, and amine bond in any prodrug forms of compounds identified by the disclosed method can be readily cleaved in vivo. Therefore, prodrug compositions can be hydrolyzed to the corresponding acid forms in plasma. For example, (2S,4R)-4-methyl glutamic acid dimethyl ester (2) was readily hydrolyzed to generate (2S,4R)-4-methyl glutamic acid (1). In a test, 2 showed an analgesic effect similar to 1 with the added benefit of exhibiting enhanced bioavailability and longer half-life.

### Method

Disclosed is a method for inhibiting, stimulating, modulating, or regulating glutamate reuptake. The method makes use of compounds that are ligands, agonists, or antagonists of glutamate receptors. It has been discovered that such compounds can bind to glutamate transporters and affect extracellular glutamate levels by affecting transporter activity. The method basically involves administering a transporter compound to an individual in need of inhibition, stimulation, modulation, or regulation of glutamate transporters. The transporter compound is preferably administered to an individual suffering from a condition, disorder, or disease involving transport of, or activation by, excitatory amino acids.

The disclosed compounds can have a variety of effects on glutamate transporter activity including activation or inhibition. Such compounds are useful to treat various neurological diseases and conditions involving glutamate transporter and glutamate receptor activation. For example, excess extracellular glutamate is a cause of excessive activation of glutamate receptors. Stimulating glutamate reuptake by glutamate transporters can ameliorate excessive activation of glutamate receptors by reducing the extracellular glutamate concentration. Similarly, inhibiting glutamate reuptake by glutamate transporters can ameliorate insufficient activation of glutamate receptors by increasing the extracellular glutamate concentration. Prodrug forms of transporter compounds can be used as drugs.

Transporter compounds can be used to treat a variety of diseases and conditions involving EAAs. These include cognitive, learning, and memory deficits, chemical toxicity (including substance tolerance and addiction), excitotoxicity, neurodegenerative disorder (such as Huntington's disease, Parkinson's disease, and Alzheimer's disease), post-stroke sequelae, epilepsy, seizures, mood disorders (such as bipolar disorder, dysthymia, and seasonal effective disorder), depression, and pain. Neurodegenerative disorders can result from dysfunction or malfunction of the receptor and/or transporter.

Binding of transporter compounds to glutamate transporters can be determined using standard techniques. Modulation of the glutamate transporters, as demonstrated by compounds showing potent in vitro affinity for the transporter, make the compounds useful for treating human neuropsychopharmacological conditions related to EAAs. Since the transporter compounds regulate the in vitro transport of Glu, they are useful in the in vivo treatment of EAA-dependent psychosis, neurodegeneration, convulsions, pain, learning and memory deficits, and other conditions involving EAAs.

In combination, in vitro and in vivo assays are predictive of the activity of the disclosed compounds for treatment of patients. The following tests can be used to demonstrate that binding activity correlates with physiological activity, both in vitro and in vivo. The results of these tests indicate that transporter compounds will be effective clinically for treatment of a variety of disorders, many of which are listed above.

Pharmacological Models

The following specific examples of assays and models can be used to assess the activity of compounds identified using the disclosed method.

Neurodegenerative Transient Global Forebrain Ischemia

The extent of protection by a test compound in a model of brain ischemia can be assayed as described in Meldrum et al., Brain Res., 571:115, 1992, and references cited therein. Male Wistar rats (250-300 g) are anesthetized using halothane-oxygen-nitrogen mixture and both vertebral arteries are permanently occluded by electrocauterisation within the alar foraminae of the first cervical vertebra. At the same time, both common carotid arteries are isolated and atraumatic clamps placed around each one. One femoral vein is cannulated to enable the subsequent iv administration of fluid. The following day cerebral ischemia is induced in the unanaesthetised animal, by tightening the clamps around the carotid arteries for 20 min. Carotid clamping results. Body temperature is maintained at 37° C. by use of a rectal probe and hot plate. Seven days after the ischemic insult rats are sacrificed and the brains processed for light microscopy. Neuroprotection is assessed by examination of the extent of damage in the cortex and hippocampus. Compounds may be selected which are active in this model.

Neurodegenerative Permanent Focal Ischemia

The extent of protection by a test compound in a model of brain ischemia can be assayed as described by Meldrum and Smith (Stroke, 23:861, 1992), and references cited therein. Male Fisher F344 rats (210-310 g) are anesthetized with halothane-oxygen-nitrogen mixture receive a small incision between the eye and ear, the mandibular muscles are retracted to expose the orbit and zygomatic arch. A small craniotomy is made to expose the base of the middle cerebral artery. Bipolar coagulation is used to permanently occlude the artery at the base. One day after the ischemic insult rats are sacrificed and the brains processed for light microscopic examination. Lesion volume is determined by using Cavalarei's principle. Compounds may be selected which are active in this model.

Maximum Electro Shock (MES) Seizure Test

This test is to determine the extent of protection by a test compound in a seizure model. This model is described by Rogawski et al. (Epilepsy Research, 15:179-184, 1993). Male NIH Swiss mice (25-30 g) were injected ip with the test drug. The mice were subjected to a 0.2 sec, 60 Hz, 50 mA electrical stimulus delivered with corneal electrodes wetted with 0.9% saline at 15-30 min post dosing. Animals failing to show tonic hind limb extension were scored as protected. Compounds may be selected which are active in this model.

Subcutaneous Metrazol (scMET) Seizure Test

This test is to determine the extent of protection by a test compound in a seizure model. The method used is that of Chen et al. (Proc. Soc. Exp. Biol. Med., 87:334, 1954). Mice are randomly assigned to vehicle or treatment groups of 3-10 animals per group and then dosed accordingly. Metrazol (pentylenetetrazol) 90 mg/kg is administered subcutaneously (sc) at different time points (0.25, 0.5, 1, 2, 4 hr) after the treatment or control groups. The mice individually housed in clear runs and observed for the presence or absence of clonic seizure activity (>5 s duration) for 30 min after metrazol dosing. A compound is considered active if no seizure is observed. Data is analyzed using a quantal measure (protection/number tested).

Rat Mechano-Allodynia Pain Model

This test is to determine the extent of protection by a test compound to neuropathic pain sensations. With the rat standing on an elevated perforated floor, mechano-allodynia is measured by applying from beneath a graded series of von Frey hairs to the mid-plantar region of the effected paws. The hair that evokes at least one withdrawal response is designated the threshold level when compared to the sham treated nerve. Alternatively, the paw can be illuminated with a noxious radiant heat and the time to paw withdrawal is measured.

The model is described by Bennett, Neuro. Report 5, 1438-1440 (1994), and references cited therein. He measures changes in withdrawal latency after chronic constriction injury; a rat is prepared by bilaterally exposing the sciatic nerves on both thighs. On one side, loosely fitting constrictive ligatures are tied around the nerve; the other side is sham manipulated but not ligated. The model can also be used to measure increases in sensitivity and decreases in latency after injection of an irritating or pain inducing substance such as capsaicin or carrageenan.

Mouse Antidepressant Forced Swim Test

This test is to determine the extent of antidepressant activity of a test compound. The model described by Trullas et al., Eur. J. Pharm. 185, 1-10 (1990), and the references cited therein. Mice are placed individually in a cylinder filled with water at 22° C.-25° C. The duration of immobility is scored during the last four minutes of a six minute test.

Elevated Plus Maze

Compounds with antidepressant activity increase both the percentage of time and percentage of entries into the open arms of an elevated plus-maze as described by Trullos et al., “1-Aminocyclopropanecarboxylates exhibit antidepressant and anxiolytic actions in animal models,” Eur. J. Pharm., 203:379-385 (1991). A mouse is placed at the intersection of the maze arms so that its head is in the center of the platform. The mouse is then scored as being in the open or enclosed arms. Arm entries are recorded and the percentage of time in each arm, as well as the percentage of entries, are calculated. The compounds are expected to be active in this test at a dose of about 1-150 mg/kg ip.

NMDA Induced Seizures

Compounds which have anticonvulsant activity for convulsions are active in a test described by Koek et al., Mechanisms for Neuromodulation and Neuroprotection, pp 665-671, Kamenka et al., eds., NPP Books, Ann Arbor, Mich. 1992. Test compounds are injected into mice at 15 minutes or 30 minutes before an ip injection of NMDA, icv or ip, respectively. ED₅₀ is determined by comparing the percentage of mice that die after 30 minutes to a group of mice that receive NMDA alone. The compounds are expected to be active in this test at a dose of about 1-150 μg/kg icv, or 1-150 mg/kg ip.

Cocaine-Induced Hypermotility

Kainate administered locally or cocaine administered subcutaneous (s.c.) induces an increase in dopamine release in nucleus accumbens and nucleus caudatus accompanied by stereotyped behavior such as hyper-locomotion, rearing, sniffing, and grooming. Inhibition of these effects can be used to indicate that a compound useful for treating addiction.

Cocaine-Induced Convulsions

Compounds with anticonvulsant activity for cocaine-induced convulsions are active in tests described by Witkin et al., Life Sciences, 48:51-56, 1991. Male Swiss Webster mice, 10-12 weeks old, are injected with the test compound ip 30 minutes prior to an ip injection of 75 mg/kg cocaine. The occurrence of convulsions is recorded for 15 minutes following the cocaine injection and are defined as loss of righting responses for at least 5 seconds and the occurrence of clonic limb movement. The ED₅₀ dose can then be calculated. The compounds are expected to be active in this test at doses of about 1-150 mg/kg ip.

A rat mechano-allodynia pain model is described by Bennett, Neuro. Report 5:1438-1440 (1994), and references cited therein. Assays useful for assessing treatment of brain and spinal cord injuries are described by Shohami et al., J. Neurotrama 10(7):109-119 (1993), Faden, J. Neurotrama 10(7):91-100 (1993), Bruno et al., Eur. J. Pharmacology 256:109-112 (1994), Long and Skolnick, Eur. J. Pharmacology 261:295-301 (1994), and Long and Skolnick, Soc. Neuroscience Abstracts 19:619.7 (1993). A Parkinson's disease model is described by Danysz et al., J. Neural Transmission 7:155-166 (1994). Assays for assessing effects on learning and memory are describe in U.S. Pat. No. 5,428,069 (column 12). An assay for assessing anxiolytic action is described by Winslow et al., Eur. J. Pharmacology 190:11-21 (1990). Assays for assessing effects on addiction are described by Papp and Willner, Psychopharmacology 103:99-102 (1991), Spyraki et al., Psychopharmacology 79:278-283 (1983), Spyraki et al., Brain Resarch 253:185-193 (1982), Papp et al., Eur. J. Pharmacology 317:191-196 (1996), Papp et al., J. Psychopharmacology 6(3):352-356 (1992), Przegalinski et al., Pharmacology Biochemistry Behavior 54(1):73-77 (1996), Evoniuk et al., Psychopharmacology 105:125-128 (1991), and Kolesnikov et al., Life Sciences 55(18):1393-1398 (1994). Assays for assessing effects on depression are described by Przegalinski et al., Neuropharmacology 36(1):31-37 (1997), and Skolnick et al., Pharmacopsychiat. 26:23-26 (1996).

The disclosed compounds act on glutamate transporters. There are five subtypes of glutamate transporters. The five subtypes of glutamate transporters demonstrate discrete cellular and regional localizations as well as distinct molecular and pharmacological characteristics (Table 1).

The information in Table 1 is adapted from Arriza et al., Proc. Natl. Acad. Sci. U.S.A., 94: 4155-4160 (1997); Gegelashvili and Schousboe, Mol. Pharmacol., 52: 6-15 (1997); Shimamoto et al., Mol. Pharmacol., 53: 195-201 (1998); Vandenberg, Clin. Exp. Pharmacol. Physiol., 25: 393-400 (1998); and Bridges et al., Curr. Pharmaceut. Des., 5: 363-379 (1999). αAA is L-α aminoadipate, CCG-III is (2S,3S,4R)-2-(carboxycyclopropyl) glycine, DHK is L-dihydrokainate, KA is kainate, 3MG is (±)-threo-3-methylglutamate, MPDC is L-anti-endo-3,4-methanopyrrolidine dicarboxylate, PDC is L-trans-pyrrolidine-2,4-dicarboxylate, SOS is L-serine-O-sulphate, T4HG is L-threo-4-hydroxyglutamate, and TBOA is DL-threo-β-hydroxyasparate.

TABLE 1

| | GLAST | GLT1 | EAAC1 | | |
|---|---|---|---|---|---|
| Transporter | (EAAT1) | (EAAT2) | (EAAT3) | EAAT4 | EAAT5 |
| Major cell type | Glia | Glia | Neurones | Neurones | ? |
| CNS distribution | Widespread | Widespread | Widespread | Cerebellum | Retina |
| Non-transportable | TBOA | MPDC, | TBOA | | THA, PDC |
| blockers | | TBOA, | | | |
| | | DHK, | | | |
| | | 3MG, KA | | | |
| Competitive | CCG-III, | CCG-III, | CCG-III, | THA, PDC, | |
| inhibitors | THA, | PDC, THA, | THA, | αAA | |
| (substrates) | MPDC, | T4HG, SOS, | MPDC, | | |
| | PDC, SOS, | ACBD | PDC, SOS, | | |
| | T4HG, | | ACBD | | |
| | ACBD | | | | |

GLAST (the rodent homologue of the human EAAT1) is predominantly located in cerebellar Bergmann glia, but is also present in glial cells throughout the central nervous system (CNS) and transiently in a small number of neurones (Storck, et al., 1992, Proc. Natl. Acad. Sci. U.S.A. 89: 10955-10959; Furuta et al., J. Neurosci., 17: 8363-8375 (1997)). GLT1 (EAAT2) is normally present exclusively in glia, and is widespread and abundant throughout the forebrain, cerebellum and spinal cord (Pines, et al., 1992, Nature 360: 464-467; Furuta et al., 1997). The transporters EAAC1 (EAAT3) (Kanai and Hediger, 1992, Nature 360: 467-471) and EAAT4 (Fairman, et al., 1995, Nature 375: 599-603) are found predominantly in neurones, with EAAC 1 being abundantly expressed throughout the brain and in spinal cord, and EAAT4 unique to Purkinje cells of the cerebellum (Furuta et al., 1997). EAAT5 is expressed predominantly in the retina, but its cellular specificity is yet to be determined (Arriza et al., 1997). The majority of synapses in the CNS are in close apposition with glial cells, and the glial transporters GLAST and GLT1 appear to be responsible for most Glu transport in the CNS (Bergles and Jahr, 1998, J. Neurosci. 18: 7709-7716; Rothstein et al., 1996). Glutamate transporters are generally described in U.S. Pat. Nos. 6,020,479 and 5,739,284. EAAT1 is described in U.S. Pat. Nos. 5,919,699, 5,932,424, and 6,100,085. EAAT2 is described in U.S. Pat. Nos. 5,658,782, 5,840,516, and 5,919,628. EAAT3 is described in U.S. Pat. Nos. 5,776,774 and 6,074,828. EAAT4 is described in U.S. Pat. Nos. 5,912,171, 6,060,307, and 6,090,560. EAAT5 is described in U.S. Pat. Nos. 5,882,926 and 5,989,825.

Radiolabelled analogues of Glu, have been used (under conditions in which they do not interact with Glu receptors) to identify and characterize Glu transporters. Thus, [³H]-Glu, [³H]-L-aspartate and [³H]-D-aspartate have proved useful in biochemical and autoradiographic studies of GluTs (Li and Balcar, Exp. Brain Res., 97: 415-422 (1994); Bridges et al., 1999). The binding affinity of these radiolabelled compounds for the various GluTs does not, however, permit the study of individual subtypes of GluT.

4MG, a ligand of the kainate receptor, selectively binds to GLAST transporter in the presence of L-dihydrokainate (DHK) and kainate (KA) and (±)-threo-3-methylglutamate (3MG), and selectively binds to GLT1 transporter in the presence of appropriate concentrations of L-serine-O-sulphate (SOS). DHK, KA and 3MG bind to GLT1 and displaces 4MG from this transporter. DHK, KA and 3MG (at concentrations below 1 mM) do not bind GLAST leaving this transporter open for 4MG binding. SOS binds to GLAST with higher affinity than to GLT1 (Kᵢ 107 μM and 1157 μM at EAAT1 and EAAT2 respectively; Arriza, et al., 1994, J. Neurosci. 14:5559-5569) and displaces 4MG from this transporter at concentrations that do not significantly displace binding from GLT1. 4MG is a prototype of the disclosed transporter compounds.

Racemic mixtures of 4-Methyglutamate have been described. It was discovered that 4MG is a desensitizer at the low affinity kainate receptor (U.S. Pat. No. 5,731,348, Gu et al., J. Medicinal Chem. 38:2518 (1995), Zhou et al., J. Pharmacology Exper. Therapeutics 280:422-427 (1997)), but also acts as a non-transportable blocker of GLT1 (Kₘ=3.4 μM) and a competitive inhibitor of GLAST (Kₘ=54 μM)(Vandenberg et al., Mol. Pharmacol., 51: 809-815 (1997)). Carroll et al., Neurosci. Lett., 255: 71-74 (1998), and Toms et al., Neuropharmacology, 36:1483-1488 (1997), describe use of [³H]-4MG to study the distribution and properties of low affinity kainate receptors. In rabbit brain membranes, Toms et al. (1997) found two binding sites for [³H]-4MG. In contrast, Carroll et al. (1998) demonstrated that [³H]-4MG bound only to a single population of sites (low affinity kainate receptors) in sections of Macaca fascicularis monkey brain. In both studies, the pharmacological profile of binding clearly establishes that all binding is to Glu receptors.

### EXAMPLE

The binding of 4MG to brain tissue was demonstrated and the binding characteristics of 4MG to glutamate transporters were analyzed. Forebrain tissue from female Sprague Dawley rats was homogenized in HEPES buffer containing Na⁺, and membranes were washed twice in the same buffer. Binding experiments were also performed in this buffer. Initial studies determined appropriate parameters for time (15 minutes incubation at 4° C.) and drug concentration (20 nM [³H]-4MG) to be used in subsequent experiments. Following incubation of [³H]-4MG with or without other compounds, binding was terminated by rapid filtration through Whatman GF/B filter discs and washing 3 times with buffer using a Brandel cell harvester. Binding was determined by scintillation spectrometry. A full range of association, dissociation, saturation and inhibition studies was performed over varying incubation times (up to 60 min). Data were analysed using computer-assisted curve fitting. Values are expressed as mean of at least triplicate determinations and multiple experiments.

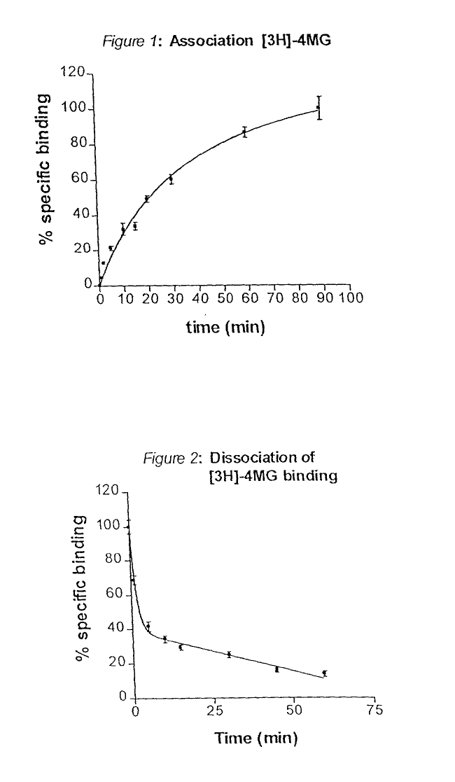

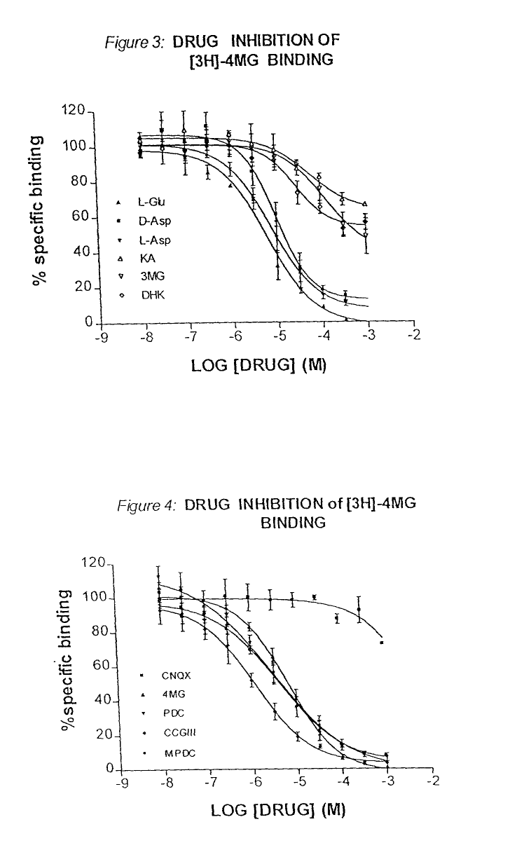

4MG was shown to bind specifically to rat brain membrane homogenates. Specific binding of 4MG (20 nM) to rat brain membrane homogenates at 4° C. represented greater than 90% of total binding (non-specific binding defined using 1 mM Glu). 4MG binding was rapid and saturable, with an association t_{½} of approximately 20 min (K_{D}=5.4 μM, Bₘₐₓ=30 pmol/mg protein; FIG. 1) and a dissociation t_{½} of approximately 3 min (FIG. 2). This binding could be completely displaced by the EAAs glutamate (IC₅₀ 6.1 μM), L-aspartate (IC₅₀ 7.5 μM), and D-aspartate (IC₅₀ 10.9 μM) (FIG. 3), and by the glutamate transporter inhibitors (2S,3S,4R)-2-(carboxycyclopropyl)glycine (CCG-III; IC₅₀ 1.2 μM), L-trans-pyrrolidine-2,4-dicarboxylate (PDC; IC₅₀ 2.9 μM), L-anti-endo-3,4-methanopyrrolidine dicarboxylate (MPDC; IC₅₀ 4.0 μM), and 4MG (IC₅₀ 6.7 μM) (FIG. 4).

Specific compounds may be used to block one of the two glial components of the binding of [³H]-4MG, allowing the study of these individual transporters (GLAST or GLT1) in isolation. L-Dihydrokainate (DHK), kainate (KA) and (±)-threo-3-methylglutamate (3MG) are known to specifically bind to only GLT1 (EAAT2) at certain concentrations, and in our experiments these compounds displace some 35-50% of [³H]-4MG binding. L-serine-O-sulphate (SOS) has reported Kᵢ values of 107 μM and 1157 μM at EAAT1 and EAAT2 respectively when these GluTs are expressed in cells of the COS-7 line (Arriza et al., 1994, J. Neurosci. 14: 5559-5569] and Kₘ values of 39 and 240 μM respectively when expressed in Xenopus oocytes (Vandenberg et al., 1998, Br. J. Pharmacol. 123: 1593-1600). SOS exhibited an IC₅₀ of 40 μM for binding of [³H]-4MG, indicating that this compound is suitable for displacing [³H]-4MG from GLAST (EAAT1), thus allowing specific binding of [³H]-4MG to GLT1 (EAAT2). 4MG displaces [³H]-4MG with a Kₘ of 3.4 μM which indicates that 4MG is a ligand of the transporter.

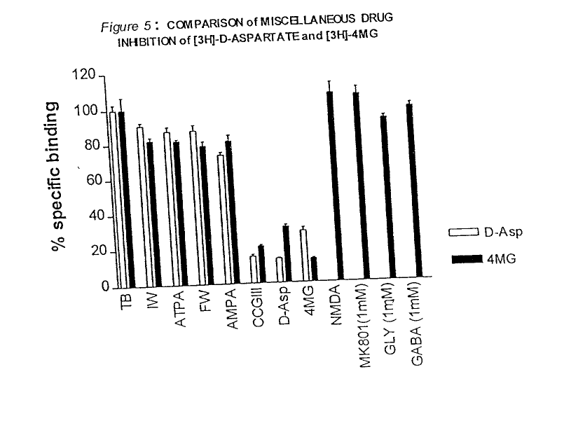

The low affinity kainate receptor antagonist 6-cyano-7-nitroquinoxaline (CNQX) did not significantly displace binding of 4MG at 100 μM (FIG. 4). In addition, agonists and antagonists at other subtypes of Glu receptor (N-methyl-D-aspartate (NMDA), (+)-5-methyl-10,11-dihydro-5H-dibenzo[a,d]cyclohepten-5,10-imine maleate (MK801), α-amino-3-hydroxy-5-methyl-4-isoxazolepropionate (AMPA), and fluorowillardiine (FW)) also failed to significantly displace 4MG binding at or above 100 μM (FIG. 5). The low affinity KA agonists ATPA and iodowillardine (IW; see Carroll et al., 1998), and the inhibitory amino acid neurotransmitters, glycine (GLY) and γ-aminobutyric acid (GABA) also failed to affect 4MG and/or D-aspartate binding to GluTs (FIG. 5).

A full range of association, dissociation, saturation and inhibition studies was performed over varying incubation times (up to 60 min). Data were analysed using computer-assisted curve fitting. Values cited are expressed as mean of at least triplicate determinations and multiple experiments.

TABLE 2
Inhibition of Binding of [³H]-4MG to Glial Glutamate Transporter

| Compounds | IC₅₀ (μM) |
|---|---|
| SYM 2031 | 85 |
| SYM 2033 | 82 |
| SYM 2034 | 63 |
| SYM 2038 | 31 |
| SYM 2049 | 42 |
| SYM 2051 | 29 |
| SYM 2061 | 13 |
| SYM 2062 | 4 |
| SYM 2064 | 6 |
| SYM 2081 | 29 |
| SYM 2082 | 73 |
| SYM 2083 | 44 |
| SYM 2115 | 16 |
| SYM 2116 | 86 |
| SYM 2133 | 42 |
| SYM 2194 | 56 |

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A method of treating a disease, condition, or disorder involving glutamate levels, the method comprising administering a transporter compound to an individual exhibiting symptoms of a disease, condition, or disorder involving transport of, or activation by, excitatory amino acids.

2. The method of claim 1 wherein the transporter compound is an agonist of a glutamate receptor.

3. The method of claim 1 wherein the transporter compound is an antagonist of a glutamate receptor.

4. The method of claim 1 wherein the transporter compound is a ligand of a glutamate receptor.

5. The method of claim 1 wherein the transporter compound selectively binds to one type of glutamate transporter.

6. The method of claim 1 wherein the transporter compound has the structure 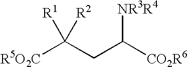
wherein
R¹, R², R⁵ and R⁶ are independently
1) C1-C6-alkyl,
2) C3-C4-alkenyl,
3) C3-C5-cycloalkyl,
4) H, or
5) halogen;
R³ and R⁴ are independently
1) H,
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) C1-C6-alkyl-SO₂—
9) CF₃SO₂—
10) PhSO₂—
11) HCO—, or
12) C3-C6-alkynyl; and
R³ and R⁴ taken together can be—CH₂(CH₂)ₙCH₂—wherein n is 0, 1, 2, or 3.

7. The method of claim 1 wherein the transporter compound has the structure 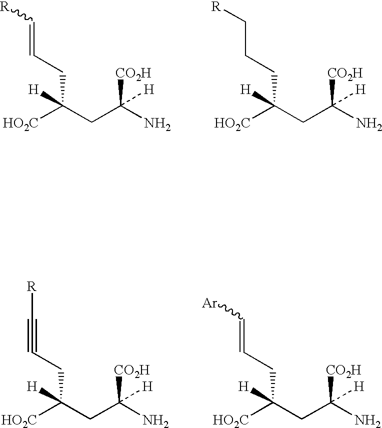
wherein R═H, C1-C6-alkyl, C3-C4-alkenyl, C3-C5-cycloalkyl, C1-C6-alkyl-CO—, C1-C6-alkyl-OCO—, C1-C6-alkyl-NHCO—, HCO—, or C3-C6-alkynyl.

8. The method of claim 1 wherein the transporter compound has the structure 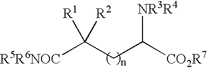
wherein
n is an integer selected from the group consisting of 0, 1, 2, and 3;
R¹, R², R⁵ and R⁷ are independently
1) C1-C6-alkyl,
2) C3-C4-alkenyl,
3) C3-C5-cycloalkyl,
4) H, or
5) halogen;
R³ and R⁴ are independently
1) H,
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) C1-C6-alkyl-SO₂—
9) CF₃SO₂—
10) PhSO₂—
11) HCO—, or
12) C3-C6-alkynyl;
R³ and R⁴ taken together can be—CH₂(CH₂)ₘCH₂—wherein m is 0, 1, 2, or 3;
R⁶ is independently
1) H,
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) C1-C6-alkyl-SO₂—
9) CF₃SO₂—
10) PhSO₂—
11) HCO—, or
12) C3-C6-alkynyl; and
R⁵ and R⁶ taken together can be—CH₂(CH₂)ₖCH₂—wherein k is 0, 1, 2, or 3.

9. The method of claim 1 wherein the transporter compound has the structure 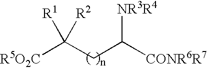
wherein
n is an integer selected from the group consisting of 0, 1, 2, and 3;
R¹, R², R⁵ and R⁷ are independently
1) C1-C6-alkyl,
2) C3-C4-alkenyl,
3) C3-C5-cycloalkyl,
4) H, or
5) halogen;
R³ and R⁴ are independently
1) H,
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) C1-C6-alkyl-SO₂—
9) CF₃SO₂—
10) PhSO₂—
11) HCO—, or
12) C3-C6-alkynyl;
R³ and R⁴ taken together can be—CH₂(CH₂)ₘCH₂—wherein m is 0, 1, 2, or 3.
R⁶ is independently
1) H,
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) C1-C6-alkyl-SO₂—
9) CF₃SO₂—
10) PhSO₂—
11) HCO—, or
12) C3-C6-alkynyl; and
R⁶ and R⁷ taken together can be—CH₂(CH₂)ₖCH₂—wherein k is 0, 1, 2, or 3.
